(19) United States
(12) Patent Application Publication
(10) Pub. No.: US 2026/0215952 A1
(43) Pub. Date: Jul. 30, 2026

(54) EXPANDING TREATMENT DEVICE FOR NASAL CAVITY AND PARANASAL SINUSES

(71) Applicant: Sung Wan LEE, Osan-si (KR)

(72) Inventor: Sung Wan LEE, Osan-si (KR)

(21) Appl. No.: 19/146,794

(22) PCT Filed: Dec. 12, 2023
(86) PCT No.: PCT/KR2023/020446

§ 371 Date: Jul. 9, 2025

(30) Foreign Application Priority Data
Jan. 10, 2023 (KR) 20-2023-0000072

(51) Int. Cl. A61F 5/08

(52) U.S. Cl. CPC A61F 5/08

(57) ABSTRACT

The present invention relates to an expanding treatment device for the nasal cavity and paranasal sinuses. The disclosed expanding treatment device for the nasal cavity and paranasal sinuses comprises a nasal insertion member, a treatment device body member, and an expanding treatment member, and thus is advantageous in that a treatment site of the nasal cavity and paranasal sinuses, requiring stimulation, can be accurately stimulated and treated using a simple procedure while causing relatively little pain.

### TECHNICAL FIELD

The present invention relates to an expanding treatment device for a nasal cavity and paranasal sinuses.

### BACKGROUND ART

A nasal cavity of a human body refers to an empty space inside a nose at a center of a face, in which an anterior portion of the nasal cavity communicates with an outer world by an external nostril, and a posterior portion of the nasal cavity communicates with a pharynx by a posterior nostril.

A paranasal sinus of the human body refers to an empty space inside a facial bone around the nose, in which the paranasal sinus is connected to the nasal cavity through a small hole, the nasal cavity is ventilated through the paranasal sinus, and secretions inside the paranasal sinus are discharged to an outside of the face through the nasal cavity. When sinusitis occurs, that is, inflammation occurs in the paranasal sinus, nasal mucus may become stagnant without being discharged, which causes symptoms such as headache, nasal congestion, and decreased concentration.

The sinusitis is treated by transmitting stimulation to the paranasal sinuses and the nasal cavity connected to the paranasal sinuses. As an example of such a treatment device, Korean Utility Model Registration No. 20-0482235 (Registration Date: Dec. 27, 2016, Invention Title: Paranasal Sinus Expansion Treatment Device) may be presented.

However, according to a conventional treatment device for a nasal cavity and paranasal sinuses, an end portion of the treatment device for the nasal cavity and the paranasal sinuses may be expanded so as to stimulate the nasal cavity and the paranasal sinuses. However, even though the nasal cavity and the paranasal sinuses are separated from each other, since a length of the treatment device for the nasal cavity and the paranasal sinuses is fixed, a treatment site may not be smoothly stimulated. In addition, when a relatively deep portion of the paranasal sinus is treated, the treatment device for the nasal cavity and the paranasal sinuses has to be inserted into a deep portion of the nasal cavity, which causes pain during the insertion while causing wounds inside the nose, so that a treatment inside the nose has to be performed in parallel.

### DISCLOSURE

### Technical Problem

One object of the present invention is to provide an expanding treatment device for a nasal cavity and paranasal sinuses, capable of accurately stimulate and treat a treatment site of the nasal cavity and the paranasal sinuses, which requires stimulation, by using a simple procedure while causing little pain.

### Technical Solution

According to one aspect of the present invention, an expanding treatment device for a nasal cavity and paranasal sinuses includes: a nasal insertion member inserted into an anterior portion of the nasal cavity of a human body; a treatment device main body member connected to the nasal insertion member, and having an inside that communicates with an inside of the nasal insertion member; and an expanding treatment member penetrating the nasal insertion member and the treatment device main body member together while being connected to the treatment device main body member, and having an end portion penetrating the nasal insertion member and expanded outward by air supplied from an outside, wherein the treatment device main body member is connected to the nasal insertion member such that a connected position is variable, and as the connection position of the treatment device main body member with respect to the nasal insertion member varies, a length of a portion of the expanding treatment member penetrating the nasal insertion member and connected to the treatment device main body member is variable, and since the length of the portion of the expanding treatment member that penetrates the nasal insertion member is variable, a position of an end portion of the expanding treatment member with respect to the nasal insertion member is also variable, so that the end portion of the expanding treatment member expanded by air applied from the outside is able to selectively stimulate at least one of a portion of the nasal cavity that is relatively deeper than a portion into which the nasal insertion member is inserted, a paranasal sinus, a sphenoid bone, and a temporomandibular joint.

### Advantageous Effects

According to an expanding treatment device for a nasal cavity and paranasal sinuses of one aspect of the present invention, the expanding treatment device may include a nasal insertion member, a treatment device main body member, and an expanding treatment member, so that a treatment site of the nasal cavity and the paranasal sinuses, which requires stimulation, can be accurately stimulated and treated using a simple procedure while causing relatively little pain.

### DESCRIPTION OF DRAWINGS

FIG. 1 is a perspective view showing an appearance of an expanding treatment device for a nasal cavity and paranasal sinuses according to a first embodiment of the present invention.

FIG. 2 is a sectional view showing the expanding treatment device for the nasal cavity and the paranasal sinuses according to the first embodiment of the present invention, which is taken vertically.

FIG. 3 is a sectional view showing a state in which an expanding part of an expanding treatment member that constitutes the expanding treatment device for the nasal cavity and the paranasal sinuses according to the first embodiment of the present invention is expanded.

FIG. 4 is a sectional view showing a state in which the expanding treatment device for the nasal cavity and the paranasal sinuses according to the first embodiment of the present invention is disassembled.

FIG. 5 is a sectional view showing a state in which a nasal insertion member and a treatment device main body member that constitute an expanding treatment device for a nasal cavity and paranasal sinuses according to a second embodiment of the present invention are connected to each other.

FIG. 6 is an enlarged view showing a separation prevention member that constitutes an expanding treatment device for a nasal cavity and paranasal sinuses according to a third embodiment of the present invention.

FIG. 7 is an enlarged view showing a state in which an expanding treatment member is attached to the separation prevention member that constitutes the expanding treatment device for the nasal cavity and the paranasal sinuses according to the third embodiment.

FIG. 8 is an enlarged view showing a nasal insertion side pressing member that constitutes the expanding treatment device for the nasal cavity and the paranasal sinuses according to the third embodiment.

FIG. 9 is an enlarged view showing a portion of the nasal insertion side pressing member that constitutes the expanding treatment device for the nasal cavity and the paranasal sinuses according to the third embodiment.

### BEST MODE

Hereinafter, an expanding treatment device for a nasal cavity and paranasal sinuses according to embodiments of the present invention will be described with reference to the drawings.

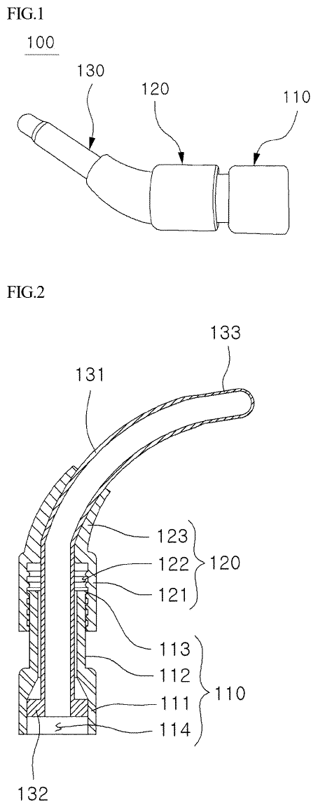

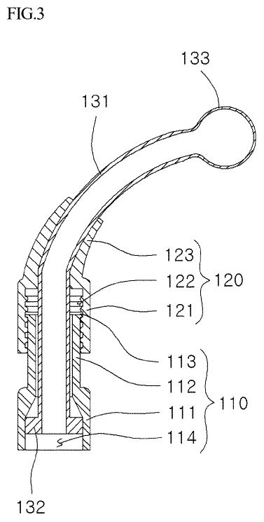

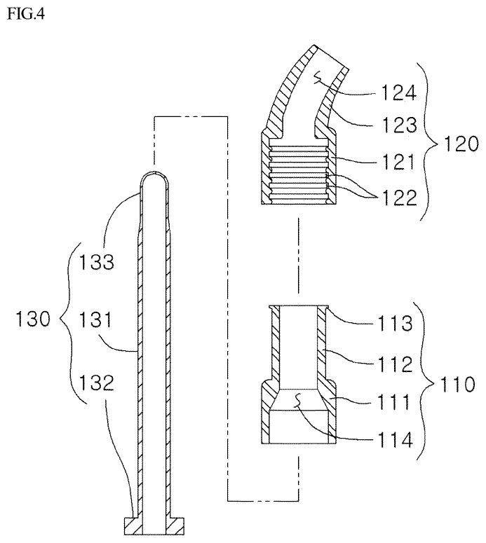

FIG. 1 is a perspective view showing an appearance of an expanding treatment device for a nasal cavity and paranasal sinuses according to a first embodiment of the present invention, FIG. 2 is a sectional view showing the expanding treatment device for the nasal cavity and the paranasal sinuses according to the first embodiment of the present invention, which is taken vertically, FIG. 3 is a sectional view showing a state in which an expanding part of an expanding treatment member that constitutes the expanding treatment device for the nasal cavity and the paranasal sinuses according to the first embodiment of the present invention is expanded, and FIG. 4 is a sectional view showing a state in which the expanding treatment device for the nasal cavity and the paranasal sinuses according to the first embodiment of the present invention is disassembled.

Referring to FIGS. 1 to 4 together, an expanding treatment device 100 for a nasal cavity and paranasal sinuses according to the present embodiment may be for expanding and treating the nasal cavity and the paranasal sinuses, which are deeper portions than the nasal cavity, while being inserted into the nasal cavity of a human body, and may include a nasal insertion member 120, a treatment device main body member 110, and an expanding treatment member 130.

The treatment device main body member 110 may be connected to the nasal insertion member 120 such that a connection position may vary, and as the connection position of the treatment device main body member 110 with respect to the nasal insertion member 120 varies, a length of a portion of the expanding treatment member 130 penetrating the nasal insertion member 120 and connected to the treatment device main body member 110 may vary.

Since the length of the portion of the expanding treatment member 130 that penetrates the nasal insertion member 120 may vary, a position of an end portion of the expanding treatment member 130 with respect to the nasal insertion member 120 may also vary, so that the end portion of the expanding treatment member 130 expanded by air applied from an outside may selectively stimulate at least one of a portion of the nasal cavity that is relatively deeper than a portion into which the nasal insertion member 120 is inserted, a paranasal sinus, a sphenoid bone, and a temporomandibular joint, which will be described in detail below.

The nasal insertion member 120 may be inserted into an anterior portion of the nasal cavity of the human body so as to fix the expanding treatment device 100 for the nasal cavity and the paranasal sinuses.

The nasal insertion member 120 may include a nasal insertion body 121, a connection groove 122, a curved part 123, and a nasal insertion side through-hole 124.

The nasal insertion body 121 may be formed in a shape of a cylinder having a predetermined length and a hollow inside.

The connection groove 122 may be recessed in an annular shape in an inner surface of the nasal insertion body 121, a plurality of connection grooves 122 may be formed at different heights, and a connection flange 113 that constitutes the treatment device main body member 110 and will be described below may be inserted into the connection groove 122 for the connection of the nasal insertion member 120 and the treatment device main body member 110.

The connection flange 113 may be inserted into one of the connection grooves 122 so as to be connected, and as a connection position of the connection flange 113 among the connection grooves 122 varies, a total length of the nasal insertion member 120 and the treatment device main body member 110 may vary.

The curved part 123 may extend from an end of the nasal insertion body 121, may have a curved shape having a predetermined curvature, and may gradually narrow toward an end of the curved part 123.

The curved part 123 may be inserted into the anterior portion of the nasal cavity of the human body, and since the curved part 123 is formed as described above, the end of the curved part 123 may be inserted into the nasal cavity of all people regardless of a type of a human body, for example, gender or age.

The nasal insertion side through-hole 124 may be formed through insides of the nasal insertion body 121 and the curved part 123, and the treatment device main body member 110 and the expanding treatment member 130 may be inserted into the nasal insertion side through-hole 124.

The treatment device main body member 110 may be connected to the nasal insertion member 120, may have an inside that communicates with an inside of the nasal insertion member 120, and may include a housing 111, an extension part 112, the connection flange 113, and a treatment device main body side through-hole 114.

The housing 111 may be formed in a shape of a cylinder having a predetermined length and a hollow inside, and may form an outer frame of the treatment device main body member 110.

The extension part 112 may extend from an end of the housing 111 by a predetermined length in a longitudinal direction of the housing 111, and may have a diameter that is relatively smaller than a diameter of the housing 111.

The connection flange 113 may extend outward from an end of the extension part 112 that is opposite to a portion in which the housing 111 is formed, and the connection flange 113 may be inserted into the connection groove 122 so that the treatment device main body member 110 may be rotatably connected to the nasal insertion member 120.

The connection flange 113 may be latched to one of the connection grooves 122 so that the treatment device main body member 110 and the nasal insertion member 120 may be connected to each other.

In addition, the connection flange 113 may be inserted into one of the connection grooves 122, and the total length of the nasal insertion member 120 and the treatment device main body member 110 may vary according to the connection position of the connection flange 113 with respect to the connection groove 122.

In detail, when the connection flange 113 is inserted into the connection groove 122 that is located relatively far from an inlet among the connection grooves 122, a protruding length of the treatment device main body member 110 that protrudes from the nasal insertion member 120 may become relatively small, and when the connection flange 113 is inserted into the connection groove 122 that is located relatively close to the inlet among the connection grooves 122, the protruding length of the treatment device main body member 110 that protrudes from the nasal insertion member 120 may become relatively long, so that the total length of the nasal insertion member 120 and the treatment device main body member 110 connected to each other may vary.

The connection flange 113 may be inserted into one of the connection grooves 122 by an external force, and at this time, the external force may be a hand of a treatment patient to which the expanding treatment device 100 for the nasal cavity and the paranasal sinuses is used, a tool, or the like.

The treatment device main body side through-hole 114 may be formed through insides of the housing 111 and the extension part 112, and the expanding treatment member 130 may be inserted through the treatment device main body side through-hole 114.

A portion of the treatment device main body side through-hole 114 that is close to an upper side of the housing 111 may have a predetermined diameter, and a portion of the treatment device main body side through-hole 114 that is close to a lower side of the housing 111 may have a diameter that is relatively larger than the diameter of the portion close to the upper side of the housing 111.

The expanding treatment member 130 may penetrate the nasal insertion member 120 and the treatment device main body member 110 together while being connected to the treatment device main body member 110, and an end portion of the expanding treatment member 130 that penetrates the nasal insertion member 120 may be expanded outward by air supplied from the outside.

In detail, the expanding treatment member 130 may include an expanding treatment body 131, an expanding latcher 132, and an expanding part 133.

The expanding treatment body 131 may be formed in a shape of a cylinder having a predetermined length, and may be inserted into the treatment device main body side through-hole 114.

The expanding latcher 132 may extend outward from an end of the expanding treatment body 131, and may be latched to an inside of the housing 111 so that a state in which the expanding treatment member 130 is connected to the treatment device main body member 110 may be maintained when the expanding treatment member 130 is inserted into the main body side through-hole.

When the expanding treatment body 131 is inserted into a portion of the treatment device main body side through-hole 114 that is close to the upper side of the housing 111, the expanding latcher 132 may be inserted into the portion of the treatment device main body side through-hole 114 that is close to the lower side of the housing 111.

Accordingly, while the expanding treatment body 131 is inserted into the treatment device main body side through-hole 114, the expanding latcher 132 may be latched to the portion of the treatment device main body side through-hole 114 that is close to the upper side of the housing 111, so that the insertion of the expanding treatment member 130 into the treatment device main body member 110 may be stopped.

The expanding part 133 may extend from an end portion of the expanding treatment body 131 that is opposite to a portion in which the expanding latcher 132 is formed, may have a relatively thin thickness as compared with the expanding treatment body 131, and may be inflated outward by the air supplied from the outside.

The expanding part 133 may be expanded by the air so as to stimulate at least one of the portion of the nasal cavity that is relatively deeper than the portion into which the nasal insertion member 120 is inserted, which is a treatment site that requires an expanding treatment, the paranasal sinus, the sphenoid bone, and the temporomandibular joint.

Since the expanding treatment device 100 for the nasal cavity and the paranasal sinuses is configured as described above, while the expanding treatment member 130 is inserted into the treatment device main body side through-hole 114, when the treatment patient using the expanding treatment device 100 for the nasal cavity and the paranasal sinuses applies an external force to an outside of the housing 111 with the hand or the like so as to rotate the housing 111 at a predetermined angle, the expanding treatment member 130 may also be rotated at a predetermined angle together with the treatment device main body member 110.

Accordingly, since the connection flange 113 is inserted into one of the connection grooves 122 so as to be connected, a protruding length of the expanding treatment member 130 that protrudes from the nasal insertion member 120 after penetrating the nasal insertion member 120 may vary, and the treatment device main body member 110 and the expanding treatment member 130 may be rotated at the predetermined angle by the external force while the connection flange 113 is inserted into one of the connection grooves 122. Accordingly, the expanding part 133 may be accurately moved to at least one of the nasal cavity, the paranasal sinus, the sphenoid bone, and the temporomandibular joint, which are treatment sites formed at different positions, and the treatment site may be stimulated, so that the expanding treatment for the treatment site may be efficiently performed.

As described above, the expanding treatment device 100 for the nasal cavity and the paranasal sinuses may include the nasal insertion member 120, the treatment device main body member 110, and the expanding treatment member 130, so that a treatment site of the nasal cavity and the paranasal sinuses, which requires stimulation, may be accurately stimulated and treated using a simple procedure while causing relatively little pain.

### MODE FOR INVENTION

Hereinafter, an expanding treatment device for a nasal cavity and paranasal sinuses according to other embodiments of the present invention will be described with reference to the drawings. In performing such description, any description that overlaps the content already disclosed above in one embodiment of the present invention will be substituted and omitted here.

FIG. 5 is a sectional view showing a state in which a nasal insertion member and a treatment device main body member that constitute an expanding treatment device for a nasal cavity and paranasal sinuses according to a second embodiment of the present invention are connected to each other.

Referring to FIG. 5 together, an expanding treatment device for a nasal cavity and paranasal sinuses according to the present embodiment may include a nasal insertion member, a treatment device main body member, and an expanding treatment member.

The nasal insertion member may be inserted into an anterior portion of the nasal cavity of a human body, and may include a nasal insertion body 221, a connection groove, a curved part, and a nasal insertion side through-hole.

The connection groove may be recessed in an annular shape in an inner surface of the nasal insertion body 221, and a plurality of connection grooves may be formed at different heights.

A rotation angle adjustment groove 225 may be recessed further in an inner wall surface of the connection groove, and a plurality of rotation angle adjustment grooves 225 may be formed in each of the connection grooves while being spaced apart from each other at a predetermined angle in a circumferential direction of the connection groove.

The treatment device main body member may be connected to the nasal insertion member, may have an inside that communicates with an inside of the nasal insertion member, and may include a housing, an extension part, a connection flange 213, and a treatment device main body side through-hole.

A rotation angle adjustment protrusion 215 may extend further outward from the connection flange 213, and a plurality of rotation angle adjustment protrusions 215 may be formed on the connection flange 213 while being spaced apart from each other at a predetermined angle.

When the connection flange 213 is inserted into one of the connection grooves, the rotation angle adjustment protrusion 215 may be inserted into the rotation angle adjustment groove 225.

In the above state, when a treatment patient using the expanding treatment device for the nasal cavity and the paranasal sinuses holds the nasal insertion member and rotates the treatment device main body member by using a hand or a tool, the rotation angle adjustment protrusions 215 may be rotated at a predetermined angle so as to be moved to adjacent rotation angle adjustment grooves 225, respectively.

With the configuration as described above, the treatment device main body member may be prevented from being arbitrarily rotated while the treatment device main body member is inserted into the nasal insertion member, and a rotation angle of the treatment device main body member with respect to the nasal insertion member, which is how much the treatment device main body member is rotated with respect to the nasal insertion member, may be adjusted.

The expanding treatment member may be connected to the treatment device main body member such that the expanding treatment member may penetrate the treatment device main body member and the nasal insertion member together while the nasal insertion member and the treatment device main body member are connected to each other, an end portion among portions of the expanding treatment member that penetrate the nasal insertion member may be expanded outward, and the expanding treatment member may include an expanding treatment body, an expanding latcher, and an expanding part.

Hereinafter, an expanding treatment device for a nasal cavity and paranasal sinuses according to a third embodiment of the present invention will be described with reference to the drawings. In performing such description, any description that overlaps the content already disclosed above in the first and second embodiments of the present invention will be substituted and omitted here.

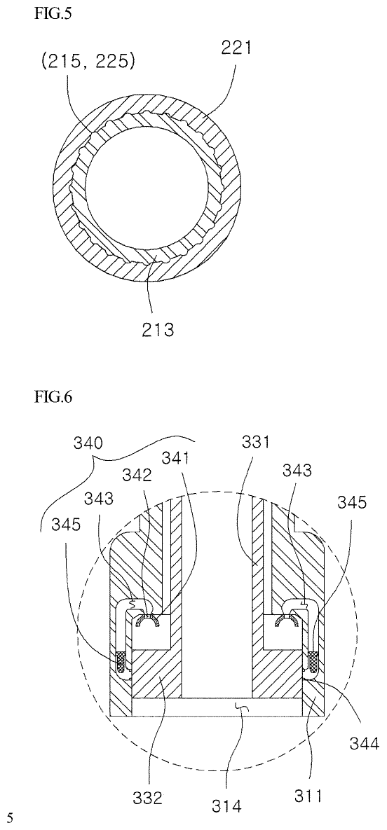

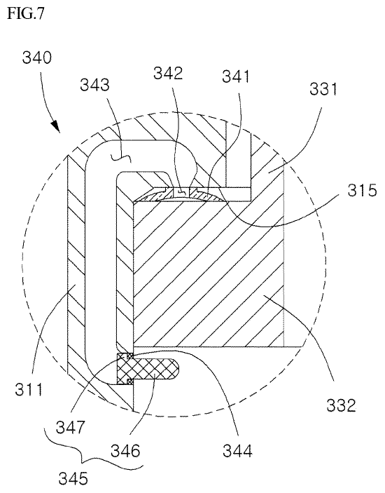

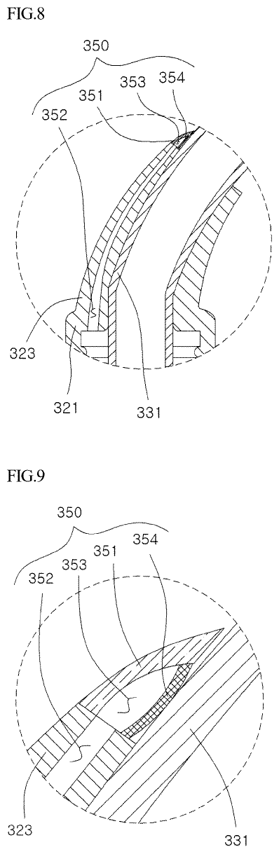

FIG. 6 is an enlarged view showing a separation prevention member that constitutes an expanding treatment device for a nasal cavity and paranasal sinuses according to a third embodiment of the present invention, FIG. 7 is an enlarged view showing a state in which an expanding treatment member is attached to the separation prevention member that constitutes the expanding treatment device for the nasal cavity and the paranasal sinuses according to the third embodiment, FIG. 8 is an enlarged view showing a nasal insertion side pressing member that constitutes the expanding treatment device for the nasal cavity and the paranasal sinuses according to the third embodiment, and FIG. 9 is an enlarged view showing a portion of the nasal insertion side pressing member that constitutes the expanding treatment device for the nasal cavity and the paranasal sinuses according to the third embodiment.

Referring to FIGS. 6 to 9 together, an expanding treatment device for a nasal cavity and paranasal sinuses according to the present embodiment may include a nasal insertion member, a treatment device main body member, an expanding treatment member, and a separation prevention member 340.

The nasal insertion member may be inserted into an anterior portion of the nasal cavity of a human body, and may include a nasal insertion body, a connection groove, a curved part 323, and a nasal insertion side through-hole.

The treatment device main body member may be connected to the nasal insertion member, may have an inside that communicates with an inside of the nasal insertion member, and may include a housing 311, an extension part, a connection flange, and a treatment device main body side through-hole 314.

The expanding treatment member may be connected to the treatment device main body member such that the expanding treatment member may penetrate the treatment device main body member and the nasal insertion member together while the nasal insertion member and the treatment device main body member are connected to each other, an end portion among portions of the expanding treatment member that penetrate the nasal insertion member may be expanded outward, and the expanding treatment member may include an expanding treatment body 331, an expanding latcher 332, and an expanding part.

The separation prevention member 340 may prevent the expanding treatment member from being arbitrarily separated from the treatment device main body member after the expanding treatment member is inserted into the treatment device main body member.

In detail, the separation prevention member 340 may include a sucker part 341, a sucker side through-hole 342, a treatment device main body side air movement hole 343, an inner latch protrusion 344, and a separation prevention part 345.

The sucker part 341 may be disposed on an inner surface of the housing 311, may have a sectional shape of a semicircle that is convex upward, may be formed of an elastic material, and may be attached to the expanding latcher 332 when the expanding treatment member is inserted into the treatment device main body member.

In detail, the sucker part 341 may be disposed on a bottom surface of a step part formed inside the housing 311, may have a lower side that is open, and may be formed in an annular shape in a circumferential direction of the housing 311.

A plurality of sucker side through-holes 342 may be spaced apart from each other at a predetermined angle in a circumferential direction of the sucker part 341, and may be formed through a convex portion of the sucker part 341, that is, a portion of the sucker part 341 that has the highest height so as to allow an inside and an outside of the sucker side through-hole 342 to communicate with each other.

The treatment device main body side air movement hole 343 may allow an inner wall surface of the housing 311 to communicate with the sucker side through-hole 342, and may move air inside the sucker part 341 toward the inner wall surface of the housing 311 when the expanding latcher 332 is attached to the sucker part 341.

A plurality of treatment device main body side air movement holes 343 may be spaced apart from each other at a predetermined angle in the circumferential direction of the sucker part 341 so as to correspond to the sucker side through-holes 342, respectively.

The inner latch protrusion 344 may protrude from an end portion of an inner surface of the treatment device main body side air movement hole 343 that is relatively close to the inner wall surface of the housing 311 toward an inner space of the treatment device main body side air movement hole 343, and a separation prevention side latcher 347 that will be described below may be latched to the inner latch protrusion 344.

The separation prevention part 345 may be disposed inside the treatment device main body side air movement hole 343, may be moved by air that moves inside the treatment device main body side air movement hole 343 so as to protrude from an inner wall of the housing 311 by a predetermined length, and may be formed of an elastic material such as rubber.

Since the separation prevention part 345 is formed of an elastic material, the separation prevention part 345 may be moved while being bent even in a portion of the treatment device main body side air movement hole 343 that is bent at a predetermined curvature.

The separation prevention part 345 may include: a separation prevention body 346 formed in a cylindrical shape of a cylinder having a predetermined length; and the separation prevention side latcher 347 extending outward from an end of the separation prevention body 346, and latched to the inner latch protrusion 344 when the separation prevention part 345 protrudes from the inner wall of the housing 311 so as to stop the movement of the separation prevention part 345.

Since the separation prevention member 340 is configured as described above, while the expanding treatment member is inserted into the treatment device main body member, when the expanding latcher 332 is attached to the sucker part 341, the air inside the sucker part 341 may move toward the inner wall of the housing 311 through the sucker side through-hole 342 and the treatment device main body side air movement hole 343, and the separation prevention part 345 may be moved together by the movement of the air so as to protrude from the inner wall of the housing 311 by a predetermined length, so that the expanding treatment member inserted into the treatment device main body member may be prevented from being arbitrarily separated from an inside of the treatment device main body member.

Meanwhile, the expanding treatment device for the nasal cavity and the paranasal sinuses according to the present embodiment may further include a nasal insertion side pressing member 350.

The nasal insertion side pressing member 350 may be formed at an end of the nasal insertion member, and may press an outer surface of a portion of the expanding treatment member that penetrates the nasal insertion member so that the expanding treatment member may be prevented from being arbitrarily rotated by generating a frictional force on the expanding treatment member while the expanding treatment member penetrates the nasal insertion member.

In detail, the nasal insertion side pressing member 350 may include a nasal insertion side pressing body 351, a nasal insertion side air movement hole 352, a nasal insertion side air introduction hole 353, and a nasal insertion side presser 354.

The nasal insertion side pressing body 351 may extend from an end portion of the curved part 323 that constitutes the nasal insertion member, and may be formed by a predetermined angle in a circumferential direction of the curved part 323 so as to cover a portion of an end of the curved part 323.

The nasal insertion side air movement hole 352 may be formed through the curved part 323 in a longitudinal direction of the curved part 323 so as to move air in an inner space of the nasal insertion body to an outside of the curved part 323.

The nasal insertion side air introduction hole 353 may be formed inside the nasal insertion side pressing body 351 while communicating with the nasal insertion side air movement hole 352 so that internal air of the nasal insertion body moved from the inner space of the nasal insertion body through the nasal insertion side air movement hole 352 may be introduced.

A portion of the nasal insertion side air introduction hole 353 that faces the expanding treatment body 331 inserted into the nasal insertion member may be formed in a shape that is open toward the outside.

The nasal insertion side presser 354 may be installed to cover an open portion of the nasal insertion side air introduction hole 353, and may be formed of an elastic material such as rubber.

Since the nasal insertion side pressing member 350 is configured as described above, when the treatment device main body member into which the expanding treatment member is inserted is connected to the nasal insertion member, the inner space of the nasal insertion body may narrow so that the internal air of the nasal insertion body inside the nasal insertion body may be introduced into the nasal insertion side air introduction hole 353 through the nasal insertion side air movement hole 352, and inner pressure of the nasal insertion side air introduction hole 353 may be increased by the internal air of the nasal insertion body introduced into the nasal insertion side air introduction hole 353 so as to inflate the nasal insertion side presser 354 so that a portion of the expanding treatment body 331 that faces the nasal insertion side presser 354 may be pressed. Accordingly, the expanding treatment member may be prevented from being arbitrarily rotated while the expanding treatment member penetrates the nasal insertion member.

When the nasal insertion side pressing member 350 is configured as described above, the expanding treatment member may penetrate into the nasal insertion member relatively slowly so that surprise or discomfort of the treatment patient may be minimized even when an insertion length of the expanding treatment member is adjusted while the expanding treatment device for the nasal cavity and the paranasal sinuses is inserted into the nasal cavity of the treatment patient.

Although specific embodiments of the present invention have been shown and described, it will be understood by a person having ordinary skill in the art that various modifications and changes can be made to the present invention without departing from the idea and scope of the present invention as set forth in the appended claims of the utility model registration. However, it is to be clearly stated that the scope of the present invention encompasses all the modifications and changes.

### INDUSTRIAL APPLICABILITY

According to an expanding treatment device for a nasal cavity and paranasal sinuses of one aspect of the present invention, a treatment site of the nasal cavity and the paranasal sinuses, which requires stimulation, may be accurately stimulated and treated using a simple procedure while causing relatively little pain, so that industrial applicability of the expanding treatment device may be construed to be high.

1. An expanding treatment device for a nasal cavity and paranasal sinuses, the expanding treatment device comprising:
a nasal insertion member inserted into an anterior portion of the nasal cavity of a human body;
a treatment device main body member connected to the nasal insertion member, and having an inside that communicates with an inside of the nasal insertion member; and
an expanding treatment member penetrating the nasal insertion member and the treatment device main body member together while being connected to the treatment device main body member, and having an end portion penetrating the nasal insertion member and expanded outward by air supplied from an outside,
wherein the treatment device main body member is connected to the nasal insertion member such that a connected position is variable, and as the connection position of the treatment device main body member with respect to the nasal insertion member varies, a length of a portion of the expanding treatment member penetrating the nasal insertion member and connected to the treatment device main body member is variable, and
since the length of the portion of the expanding treatment member that penetrates the nasal insertion member is variable, a position of an end portion of the expanding treatment member with respect to the nasal insertion member is also variable, so that the end portion of the expanding treatment member expanded by air applied from the outside is able to selectively stimulate at least one of a portion of the nasal cavity that is relatively deeper than a portion into which the nasal insertion member is inserted, a paranasal sinus, a sphenoid bone, and a temporomandibular joint.

2. The expanding treatment device of claim 1, wherein the nasal insertion member includes:
a nasal insertion body formed in a shape of a cylinder having a predetermined length and a hollow inside;
a plurality of connection grooves recessed in an annular shape in an inner surface of the nasal insertion body, and formed at different heights;
a curved part extending from an end of the nasal insertion body, having a curved shape having a predetermined curvature, and gradually narrowing toward an end of the curved part; and
a nasal insertion side through-hole formed through insides of the nasal insertion body and the curved part.

3. The expanding treatment device of claim 2, wherein the treatment device main body member includes:
a housing formed in a shape of a cylinder having a predetermined length and a hollow inside;
an extension part extending from an end of the housing by a predetermined length, and having a diameter that is relatively smaller than a diameter of the housing;
a connection flange extending outward from an end of the extension part that is opposite to a portion in which the housing is formed; and
a treatment device main body side through-hole formed through insides of the housing and the extension part, and
the connection flange is latched to one of the connection grooves so that the treatment device main body member and the nasal insertion member are connected to each other.

4. The expanding treatment device of claim 3, wherein the expanding treatment member includes:
an expanding treatment body formed in a shape of a cylinder having a predetermined length;
an expanding latcher extending outward from an end of the expanding treatment body, and latched to an inside of the housing so that the expanding treatment member is connected to the treatment device main body member; and
an expanding part extending from an end portion of the expanding treatment body that is opposite to a portion in which the expanding latcher is formed, having a relatively thin thickness as compared with the expanding treatment body, and inflated outward by the air supplied from the outside.

5. The expanding treatment device of claim 4, further comprising:
a separation prevention member configured to prevent the expanding treatment member from being arbitrarily separated from the treatment device main body member after the expanding treatment member is inserted into the treatment device main body member,
wherein the separation prevention member includes:
a sucker part disposed on an inner surface of the housing, having a sectional shape of a semicircle that is convex upward, formed of an elastic material, and attached to the expanding latcher when the expanding treatment member is inserted into the treatment device main body member;
a sucker side through-hole formed through a convex portion of the sucker part;
a treatment device main body side air movement hole configured to allow an inner wall surface of the housing to communicate with the sucker side through-hole, and move air inside the sucker part toward the inner wall surface of the housing when the expanding latcher is attached to the sucker part;
an inner latch protrusion protruding from an end portion of an inner surface of the treatment device main body side air movement hole that is close to the inner wall surface of the housing toward an inner space of the treatment device main body side air movement hole; and
a separation prevention part disposed inside the treatment device main body side air movement hole, moved by air that moves inside the treatment device main body side air movement hole so as to protrude from an inner wall of the housing by a predetermined length, and formed of an elastic material,
the separation prevention part includes:
a separation prevention body formed in a cylindrical shape of a cylinder having a predetermined length; and
a separation prevention side latcher extending outward from an end of the separation prevention body, and latched to the inner latch protrusion when the separation prevention part protrudes from the inner wall of the housing so as to stop the movement of the separation prevention part, and
while the expanding treatment member is inserted into the treatment device main body member, when the expanding latcher is attached to the sucker part, the air inside the sucker part moves toward the inner wall of the housing through the sucker side through-hole and the treatment device main body side air movement hole, and the separation prevention part is moved together by the movement of the air so as to protrude from the inner wall of the housing by a predetermined length, so that the expanding treatment member inserted into the treatment device main body member is prevented from being arbitrarily separated from the treatment device main body member.
